(19) United States
(12) Patent Application Publication
(10) Pub. No.: US 2024/0325599 A1
(43) Pub. Date: Oct. 3, 2024

(54) LARGE APERTURE-BASED TISSUE ENGINEERING SCAFFOLD AND USE THEREOF

(71) Applicant: SHANGHAI RESTHETIC BIO CO., LTD, Shanghai (CN)

(72) Inventors: Yu LIU, Shanghai (CN); Zheng CI, Shanghai (CN)

(21) Appl. No.: 18/262,588

(22) PCT Filed: Jan. 21, 2022
(86) PCT No.: PCT/CN2022/073191

§ 371 Date: Jul. 24, 2023

(30) Foreign Application Priority Data
Jan. 22, 2021 (CN) 202110087461.7

(51) Int. Cl. A61L 27/22; A61L 27/18; A61L 27/24; A61L 27/36; A61L 27/52; A61L 27/56; A61L 27/58

(52) U.S. Cl. CPC A61L 27/222; A61L 27/18; A61L 27/225; A61L 27/227; A61L 27/24; A61L 27/3608; A61L 27/3645; A61L 27/52; A61L 27/56; A61L 27/58

(57) ABSTRACT

The present invention provides a tissue engineering scaffold. Specifically, the tissue engineering scaffold is a bio-gel-frame structure complex manufactured by uniformly filling a degradable bio-gel in a hard large aperture frame structure. The tissue engineering scaffold of the present invention optimizes the aperture of a conventional large aperture frame structure and improves the cell inoculation efficiency. In addition, the present invention also provides a preparation method for the novel tissue engineering scaffold and a use thereof in repairing hard tissue defects.

### TECHNICAL FIELD

The present invention relates to the field of biomedical tissue engineering, in particular to a tissue engineering scaffold and preparation method thereof.

### BACKGROUND

Hard tissue defects, including cartilage or bone defects, are becoming increasingly common in clinical diagnosis and treatment. The current treatment methods still rely mainly on autologous tissue transplantation, but there are problems such as high risk of infection, new donor defects, and limited donor areas. In recent years, the progress of tissue engineering has provided a new method for the treatment of various hard tissue defects. Scaffold materials, as an important part of the three elements of tissue engineering, play a crucial role in the construction of tissue engineering bone or cartilage. Therefore, finding a scaffold material with suitable material characterization, good cell compatibility, and osteogenic induction activity has become a hot topic in bone tissue engineering at present.

The existing tissue engineering scaffolds for hard tissue repair are mostly divided into two types: one is a sponge like porous scaffold (such as collagen sponge, polyglycolic acid/polylactic acid (PGA/PLA) scaffold, etc.); another type is a macroporous framework represented by decalcified bone and PCL framework. These two types of scaffolds have their own advantages and disadvantages. Sponge like porous scaffolds can be constructed with different pore sizes by adjusting solute concentration and freeze-drying parameters to meet the inoculation needs of various cells. However, the mechanical strength of these scaffolds is often unsatisfactory and cannot meet the strength requirements of immediate repair. Scaffolds represented by decalcified bone and PCL framework may have good mechanical strength, meeting the mechanical strength requirements for immediate repair of various sites. However, the pore size of that is difficult to accurately control, and excessive pore size leads to the inability to effectively load cells when inoculating cell suspensions, resulting in low cell seeding efficiency. Especially for materials from natural sources such as decalcified bone matrix, their pore sizes may vary depending on the batch of materials, and may affect the overall tissue regeneration effect.

Therefore, there is an urgent need to develop a tissue engineering scaffold with suitable pore size and good mechanical strength in this field.

### SUMMARY OF THE INVENTION

The purpose of the present invention is to provide a tissue engineering scaffold suitable for the repair of hard tissue defects.

The first aspect of the present invention provides a tissue engineering scaffold, which comprises:
(a) a hard large aperture frame structure; and
(b) degradable bio-gel loaded or filled in the hard large aperture frame structure.

In another preferred embodiment, the degradable bio-gel is selected from the group consisting of gelatin, collagen, silk fibroin, hydrogel, and a combination thereof.

In another preferred embodiment, the hard large aperture frame structure has a certain degree of hardness and mechanical strength.

In another preferred embodiment, the hard large aperture frame structure is made of degradable biomaterials.

In another preferred embodiment, the hard large aperture frame structure is selected from the group consisting of a decalcified bone matrix, a PCL framework, and a combination thereof.

In another preferred embodiment, the hard large aperture frame structure has an aperture of 300-800 μm, and a porosity of 80%-90%.

In another preferred embodiment, the hard large aperture frame structure is a decalcified bone matrix.

In another preferred embodiment, the decalcified bone matrix is derived from an allogeneic bone repair material.

In another preferred embodiment, the decalcified bone matrix is derived from a xenogeneic bone repair material.

In another preferred embodiment, the shape of the decalcified bone matrix comprises a cylinder, a cuboid or other specific shape.

In another preferred embodiment, the decalcified bone matrix has a thickness of 0.3-0.8 cm, preferably 0.4-0.6 cm, and most preferably 0.5 cm.

In another preferred embodiment, the decalcification amount of the decalcified bone matrix is 30% to 50%.

In another preferred embodiment, the decalcified bone matrix has an aperture of 300-800 m and a porosity of 80%-90%.

In another preferred embodiment, the aperture size of the tissue engineering scaffold can be adjusted by the concentration of loaded bio-gel and the time of freeze-drying treatment.

In another preferred embodiment, the tissue engineering scaffold can also be loaded with chondrocyte suspension containing chondrocytes, cartilage gel or cartilage sheet pieces.

In another preferred embodiment, the chondrocytes are derived from a human or non-human mammal.

In another preferred embodiment, the chondrocytes are derived from autologous chondrocytes or allogeneic chondrocytes, preferably autologous chondrocytes.

In another preferred embodiment, the chondrocytes are derived from elastic cartilage, fibrocartilage or hyaline cartilage.

In another preferred embodiment, the chondrocytes are taken from the subject's autologous chondrocytes.

In another preferred embodiment, the autologous chondrocytes are derived from elastic cartilage, fibrocartilage or hyaline cartilage.

In another preferred embodiment, the subject is a human or a non-human mammal.

In another preferred embodiment, the subject has a hard tissue defect.

In another preferred embodiment, the hard tissue defect includes joint defect, maxillofacial cartilage and related hard tissue defect, nasal septum defect, and a combination thereof.

In another preferred embodiment, the joint defect is a knee joint defect, an elbow joint defect, a hip joint defect, an ankle joint defect, a wrist joint defect, a mandibular joint defect, and a combination thereof.

In another preferred embodiment, the concentration of chondrocytes in the chondrocyte suspension is 1.0×10⁸ cells/ml-10×10⁸ cells/ml.

In another preferred embodiment, the cartilage gel comprises a cell population of chondrocytes and extracellular matrix secreted by chondrocytes, wherein the extracellular matrix encapsulates the cell population, and the cartilage gel is in a gel state, and the density of chondrocytes is at least 1.0×10⁸ cells/ml or 1.0×10⁸ cells/g.

In another preferred embodiment, the cartilage gel is prepared by gelation culture of chondrocytes.

In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the adhesion rate of the cartilage gel is ≥90%, preferably ≥95%.

In another preferred embodiment, in the cartilage gel, the concentration of chondrocytes is 1.0×10⁸ cells/ml-10×10⁸ cells/ml, preferably 1.5-5×10⁸ cells/ml.

In another preferred embodiment, the cartilage gel is obtained by gelation culture for 2.5-5.5 days, preferably for 3-5 days.

In another preferred embodiment, the cartilage sheet pieces comprise a cell population composed of chondrocytes and extracellular matrix secreted by chondrocytes, wherein the extracellular matrix encapsulates the cell population, and the cartilage pieces are prepared by mincing the cartilage sheet, wherein the density of chondrocytes is at least 1.0×10⁸ cells/ml or 1.0×10⁸ cells/g.

In another preferred embodiment, the concentration of chondrocytes in the cartilage sheet is 1.0×10⁸ cells/ml-10×10⁸ cells/ml, preferably 1.5-5×10⁸ cells/ml.

In another preferred embodiment, the cartilage sheet is obtained by gelation culture for 6-30 days, preferably 7-20 days, and most preferably 10-15 days.

In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the thickness of the cartilage sheet is 0.2-0.25 mm.

In another preferred embodiment, the average volume of the cartilage sheet pieces is 0.2 μl.

In another preferred embodiment, the surface area of the cartilage sheet pieces is 0.05-10 mm², preferably, 1-5 mm², more preferably, the average area is 1 mm².

The second aspect of the present invention provides a method for preparing the tissue engineering scaffold of the first aspect of the present invention, comprising the steps of: loading or filling bio-gel onto a hard large aperture frame structure to obtain the tissue engineering scaffold.

In another preferred embodiment, the method comprises the steps of:
(i) preparing a bio-gel solution and place it in a centrifuge tube;
(ii) placing the hard large aperture frame structure in the centrifuge tube containing the bio-gel solution and centrifuging;
(iii) refrigerating the centrifuge tube after centrifugation, taking out the contents for freezing to obtain a bio-gel frame structure complex;
(iv) freeze-drying the bio-gel frame structure complex in vacuum to obtain a freeze-dried bio-gel frame structure complex;
(v) using chemical crosslinking agents to crosslink the freeze-dried bio-gel frame structure complex to obtain a crosslinked bio-gel frame structure complex;
(vi) rinsing the crosslinked bio-gel frame structure complex with deionized water and freeze-drying in vacuum to obtain the tissue engineering scaffold.

In another preferred embodiment, the bio-gel is medical gelatin.

In another preferred embodiment, the concentration of the bio-gel solution is 0.3%-1.0%, preferably 0.6%.

In another preferred embodiment, the hard large aperture frame structure is a decalcified bone matrix.

In another preferred embodiment, the centrifugal speed is 500 r/min and the centrifugal time is 2 minutes.

In another preferred embodiment, the centrifuged centrifuge tube is refrigerated at 2-8° C. for 5-10 hours, preferably 6-8 hours.

In another preferred embodiment, the contents are frozen at −20° C. to −30° C. for 5-10 hours, preferably 6-8 hours.

In another preferred embodiment, the bio-gel frame structure complex is freeze-dried for 10-20 hours, preferably 10-16 hours, more preferably 12-14 hours.

In another preferred embodiment, the chemical crosslinking agent is selected from the group consisting of EDC, genipin, glutaraldehyde, and a combination thereof.

The third aspect of the present invention provides a use of the tissue engineering scaffold of the first aspect of the present invention for preparing a medical product for repairing hard tissue defects.

In another preferred embodiment, the hard tissue defects include joint defect, maxillofacial cartilage and related hard tissue defect, nasal septum defect, and a combination thereof.

In another preferred embodiment, the joint defect is a knee joint defect, an elbow joint defect, a hip joint defect, an ankle joint defect, a wrist joint defect, a mandibular joint defect, and a combination thereof.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the electron microscopy and general photos of the decalcified bone matrix material. Wherein, the left one shows the electron microscope image of the pores in the decalcified bone matrix, with a scale bar of 200 μM in the image; the right one shows the general photo of the decalcified bone matrix.

FIG. 2 shows a photo of gelatin particles.

FIG. 3 shows the electron microscopy and general photos of the bio-gel decalcified bone matrix complex scaffold. Wherein, the left one shows the electron microscope structure, with a scale bar of 200 μM in the image; the right one shows the general photo.

FIG. 4 shows the cartilage like tissue regenerated after implantation into experimental animals after loading chondrocytes with the bio-gel decalcified bone matrix complex.

### DETAILED DESCRIPTION

After extensive and in-depth research, the inventor unexpectedly discovered for the first time that the bio-gel frame complex scaffold prepared by filling the hard large aperture frame structure with bio-gel can be used for repairing hard tissue defects. Experiments have shown that the bio-gel frame complex scaffold can effectively load inoculated cells and has good mechanical strength. After implantation in vivo, it can successfully regenerate into cartilage like tissue.

On this basis, the present invention has been completed.

### Term

Unless otherwise defined, all technical and scientific terms used herein have the same meanings as those commonly understood by those skilled in the art to which the present invention belongs.

As used herein, when used in reference to specific enumerated values, the term “about” means that the value may vary by no more than 1% from the enumerated value. For example, as used herein, “about 100” includes all values between 99 and 101 (e.g., 99.1, 99.2, 99.3, 99.4, etc.).

As used herein, the term “contains” or “includes (comprises)” may be open, semi-closed and closed. In other words, the term also includes “substantially consisting of . . . ” or “consisting of . . . ”.

### Bio-Gel

As used herein, “bio-gel” refers to a type of degradable biological agent that has a certain fluidity at room temperature and becomes a solid form after the temperature drops (such as to 10° C. or lower).

In the example of the present invention, the bio-gel comprises an agent selected from the group consisting of gelatin, collagen, silk fibroin, hydrogel, and a combination thereof.

In a preferred embodiment of the present invention, the bio-gel used is medical gelatin, and the medical gelatin is mixed with an aqueous solution to prepare a bio-gel aqueous solution with a concentration of 0.3%-1.0%, preferably 0.6%.

### Hard Large Aperture Frame Structure

As used herein, the term “hard large aperture frame structure” refers to a degradable biomaterial frame structure with a certain degree of hardness and mechanical strength. It can provide mechanical support when used for repairing hard tissue defects, and can be degraded in organisms without producing harmful substances. Therefore, the body has a low immune response and good biological safety.

The hard large aperture framework structure of the present invention includes (but is not limited to) decalcified bone matrix and PCL framework.

In another preferred embodiment, the hard large aperture frame structure is a decalcified bone matrix.

### Decalcified Bone Matrix

Decalcified bone matrix (DBM) is a bone graft material that can reduce immunogenicity by decalcification of allogeneic bone or xenogeneic bone. It is a kind of composite of natural bone grafting material composed of collagen, non-collagen and low concentration of growth factors (such as bone morphogenetic protein, wherein bone morphogenetic protein in bone is surrounded by dense mineral composition. Non-decalcified bone has no osteoinductive ability, and different degrees of decalcification correspond to different mechanical strength), which is mainly from skull, femoral shaft and tibial shaft of human or animals, such as pig, cattle, dog, rabbit, etc.

The decalcified bone matrix used in the preferred embodiment of the present invention has a thickness of 0.3-0.8 cm, preferably 0.4-0.6 cm, and most preferably 0.5 cm. The decalcification amount of the decalcified bone matrix is 30% to 50%, the degree of decalcification is appropriate, the supporting effect is good, and it is easy to trim and cut to a suitable shape and size. The pore size of the demineralized bone matrix is 300-800 m.

### Chemical Crosslinking Agent

In the preparation process of the tissue engineering scaffold of the present invention, a chemical crosslinking agent is used to crosslink the bio-gel frame structure complex after freeze drying.

In a preferred embodiment of the present invention, the chemical crosslinking agent is selected from EDC, genipin, or glutaraldehyde.

### Hard Tissue Defects

The tissue engineering scaffold of the present invention can be used for repairing hard tissue defects.

The hard tissue defects include, but is not limited to joint defect, maxillofacial cartilage and related hard tissue defect, nasal septum defect, and a combination thereof.

### Cartilage Gel and Preparation Thereof

The tissue engineering scaffold of the present invention can be loaded with cartilage gel containing chondrocytes.

As used herein, “gel cartilage”, “cartilage gel”, “gel-state cartilage”, or “gel-like cartilage” can be used interchangeably, all refer to the cartilage (stem) cells in gel state, in particular, chondrocytes with a specific concentration are inoculated on and/or spread on a flat or substantially flat culture surface, so that the inoculated chondrocytes form a laminated structure, and the chondrocytes having a laminated structure are cultured under suitable gelation culture conditions, thereby forming a gel-like cartilage culture.

The gel cartilage is a new type of cartilage different from free chondrocytes, centrifugally precipitated chondrocytes and cartilage pellet. The gel cartilage of the present invention can be regarded as a specific form of cartilage between free chondrocytes and dense cartilage masses. During the process of gelation culture of the gel cartilage of the present invention, the chondrocytes not only contact and/or interact with adjacent cells on the plane (X-Y plane), but also contact and/or interact with adjacent chondrocytes in multiple directions such as above and/or below and/or the upper or lower side, so as to promote the chondrocytes to secrete and form more extracellular matrix, therefore, the gelation cultured chondrocytes are wrapped in extracellular matrix with a certain viscosity, so that the gel cartilage of the present invention has a close connection, but has a certain viscosity and fluidity, so that the gel cartilage of the present invention is more suitable for inoculation and loading on various carrier materials (especially porous carrier materials), thereby forming a complex for repairing cartilage.

In addition, the gel cartilage of the present invention has a gel state on the one hand, and on the other hand, it has an unusually high cell density (usually at least 1.0×10⁸ cells/ml or more, such as 1.0×10⁸-10×10⁸ cells/ml). Therefore, it is particularly suitable for the preparation of grafts for repairing various types of cartilage, or for cartilage transplantation or cartilage repair surgery.

Preferably, in the present invention, the gel cartilage is formed by in vitro culture for a period of time t1 under the gelation culture condition. Preferably, the t1 is 2.5-5.5 days, preferably 3-5 days.

One feature of the gel cartilage of the present invention is laminated inoculation, that is, after chondrocytes with a specific density are inoculated into a culture container, the inoculated chondrocytes will form a multilayer chondrocyte group (i.e., a chondrocyte group with a laminated structure) through, for example, deposition. Typically, calculated on the basis of the culture area of the culture dish (or culture container), and assuming that the degree of confluence of the monolayer cells is 100%, the number of cells S1 of the laminated inoculation of the present invention is n times of the number of cells S0 for the degree of confluence of 100% (i.e., S1/S0=n), wherein n is 1.5-20, preferably 2-10, more preferably 2.5-5.

### Cartilage Sheet and Preparation Thereof

The tissue engineering scaffold of the present invention can be loaded with cartilage sheet pieces containing chondrocytes.

As used herein, “cartilage sheet”, “sheet-like cartilage”, or “cartilage sheet of the present invention” can be used interchangeably, all refer to the cartilage (stem) cells in sheet state, in particular, chondrocytes with a specific concentration are inoculated on and/or spread on a flat or substantially flat culture surface, so that the inoculated ear chondrocytes form a laminated structure, and the chondrocytes having a laminated structure are cultured under suitable culture conditions, thereby forming a sheet-like cartilage culture.

The “cartilage sheet” is prepared on the basis of the preparation of the “cartilage gel” of the present invention by prolonging the gelation culture time. That is, in the present invention, the chondrocytes are inoculated and/or spread on a flat or substantially flat culture surface and cultured in vitro for a period of time t2 under gelation culture conditions, thereby forming a cartilage sheet. Preferably, the t2 is 6-30 days, preferably 7-20 days, and most preferably 10-15 days.

On the one hand, the cartilage sheet of the present invention has an unusually high cell density (usually at least 1.0×10⁸ cells/ml or more, such as 1.0×10⁸-10×10⁸ cells/ml). On the other hand, its thickness is thin (only 0.2-0.25 mm) and it has good toughness, and can be cut into “cartilage sheet pieces” with an average volume of 0.2 μl, which can be filled in the porous frame structure by simple centrifugation. Therefore, it is especially suitable for preparing and repairing various types of cartilage grafts, or for cartilage transplantation or cartilage repair surgery.

As used herein, “specific concentration” or “specific density” refers to inoculating 1.0×10⁷-2.0×10⁷ cells, preferably 1.5×10⁷ cells, into a 3.5 cm culture dish (e.g., one well in a six-well plate). After gelation culture for different times, the cartilage gel containing chondrocytes with a density of 1.0×10⁸-10×10⁸ cells/ml or the cartilage sheet containing chondrocytes with a density of 1.0×10⁸-10×10⁸ cells/ml is finally formed.

In another preferred embodiment, the gelation culture condition is: inoculating chondrocytes with a specific density, and using gelation medium for culture, wherein the gelation medium is high-glucose (4-5 wt % glucose) DMEM medium containing 10% fetal bovine serum and 100 U/ml penicillin-streptomycin.

After loading the cartilage gel or cartilage sheet pieces, the tissue engineering scaffold of the present invention needs to undergo chondrogenic culture to form a graft that can be used to repair hard tissue defects. As used herein, the term “chondrogenic culture” refers to culturing a porous frame structure inoculated with cartilage gel or cartilage sheet pieces using chondrogenic medium, wherein the frame structure eventually forms an integrated cartilage gel-frame structure complex or cartilage sheet pieces-frame structure complex, i.e., the cartilage tissue engineering complex of the present invention, which can be used for transplantation into cartilage defects in human or animal bodies.

### The Medium Used in the Present Invention

Chondrogenic medium: high glucose DMEM medium, 1% 1×ITS premium (ITS universal culture mixture, containing insulin, transferrin, selenite, linoleic acid, bovine serum protein, pyruvate, ascorbic acid phosphate), 40 μg/ml proline, 10 ng/ml TGF-β 1, 100 ng/ml IGF-1, 40 ng/ml dexamethasone and 50 μg/ml vitamin C.

Gelation medium: DMEM medium containing 4-5 wt % glucose, 10% FBS (v/v) and 100 U/ml penicillin-streptomycin.

The beneficial effects of the present invention:
(1) The present invention uses compound gelatin (or collagen, silk fibroin, hydrogel, etc.) to fill the pores of decalcified bone matrix or PCL frame structure with gelatin to build a new tissue engineering scaffold. The new decalcified bone scaffold after changing the pore size is expected to provide a new method for the construction of tissue engineering bone.
(2) The tissue engineering scaffold of the present invention not only has an appropriately pore size that can effectively load inoculated cells, but also has good mechanical strength that can meet the mechanical strength requirements for immediate repair of various parts.
(3) The tissue engineering scaffold of the present invention can precisely control the pore size by adjusting the concentration ratio of bio-gel or freeze-drying parameters according to the type of cells required to be loaded.

Hereinafter, the present invention will be further described with reference to specific examples. The present invention is further explained below in conjunction with specific example. It should be understood that these examples are only for illustrating the present invention and not intend to limit the scope of the present invention. The experimental method without specific conditions in the present embodiment is generally in accordance with conventional conditions, or in accordance with the conditions recommended by the commodity manufacturer. Unless otherwise stated, percentages and parts are calculated by weight. Unless otherwise stated, the materials and reagents used in the examples of the present invention are commercially available products.

### Example 1

### Preparation of the Bio-Gel Decalcified Bone Matrix Complex Scaffold

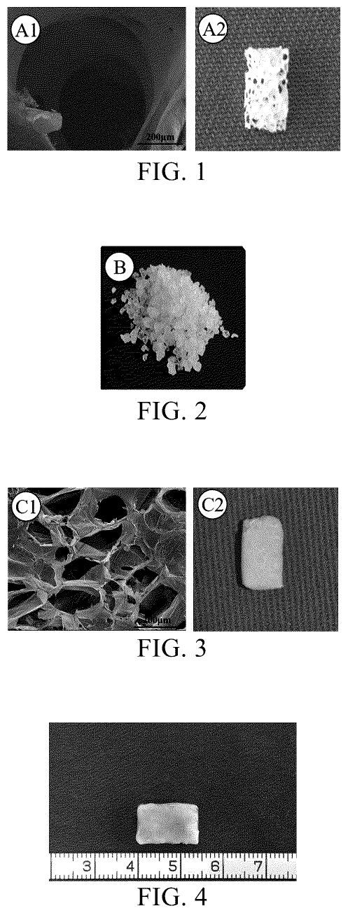

The bio-gel decalcified bone matrix complex scaffold was prepared according to the following steps:
1. Medical gelatin particles were placed in a centrifuge tube, added with deionized water, and placed in a shaker at 37° C. to prepare a 0.6% gelatin aqueous solution;
2. A decalcified bone matrix (as shown in FIG. 1) was provided, and placed in gelatin aqueous solution, then it was centrifuged at 500 r/min for 2 minutes by a centrifuge;
3. The centrifuge tube containing decalcified bone matrix and gelatin solution after centrifugation was placed in a refrigerator at 4° C. for 8 hours;
4. The centrifuge tube was taken out from the 4° C. refrigerator, the bio-gel decalcified bone matrix complex was taken out and placed in a −20° C. refrigerator for 8 hours of freezing;
5. The frozen bio-gel decalcified bone matrix complex was frozen dry for 12 hours using a vacuum dryer;
6. The decalcified bone-gelatin scaffold was immersed in a chemical cross-linking agent (EDC, genipin or glutaraldehyde) for cross-linking. After the cross-linking was completed, the residual chemical cross-linking agent was removed by repeatedly washing and soaking with deionized water, and then the final bio-gel decalcified bone matrix complex scaffold was obtained by freeze drying in vacuum (as shown in FIG. 3).

Compared with the pure decalcified bone matrix (FIG. 1), the pore size of the bio-gel decalcified bone matrix complex prepared in this example is significantly reduced (FIG. 3), which can more effectively load inoculated cells.

### Example 2

### Application of the Bio-Gel Decalcified Bone Matrix Complex Scaffold

First, chondrocytes were inoculated in a concentration of 7×10⁷ cells/ml onto the bio-gel decalcified bone matrix complex scaffold prepared in Example 1;

Then the cell scaffold complex was incubated in an incubator of 37° C., 95% humidity, and 5% carbon dioxide, and added with chondrogenic medium (high glucose DMEM medium, 1% 1×ITS premium (ITS universal culture mixture, containing insulin, transferrin, selenite, linoleic acid, bovine serum protein, pyruvate, ascorbic acid phosphate), 40 μg/ml proline, 10 ng/ml TGF-β 1, 100 ng/ml IGF-1, 40 ng/ml dexamethasone and 50 μg/ml vitamin C) was added. After 8 weeks of in vitro culture, it was transplanted back into the body; After 4 weeks of in vivo regeneration, samples were taken, and the successfully regenerated cartilage like tissue is shown in FIG. 4.

The results show that the bio-gel decalcified bone matrix complex scaffold prepared in Example 1 of the present invention effectively loaded the chondrocytes inoculated on and the complex scaffold provided good mechanical support at the defect site, which can successfully regenerate into cartilage-like tissue in vivo.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A tissue engineering scaffold, which comprises:
(a) a hard large aperture frame structure; and
(b) degradable bio-gel loaded or filled in the hard large aperture frame structure.

2. The tissue engineering scaffold of claim 1, wherein the degradable bio-gel is selected from the group consisting of gelatin, collagen, silk fibroin, hydrogel, and a combination thereof.

3. The tissue engineering scaffold of claim 1, wherein the hard large aperture frame structure is selected from the group consisting of a decalcified bone matrix, a PCL framework, and a combination thereof.

4. The tissue engineering scaffold of claim 1, wherein the hard large aperture frame structure has an aperture of 300-800 μm, and a porosity of 80%-90%.

5. The tissue engineering scaffold of claim 1, wherein the aperture size of the tissue engineering scaffold can be adjusted by the concentration of loaded bio-gel and the time of freeze-drying treatment.

6. The tissue engineering scaffold of claim 1, wherein the tissue engineering scaffold may further be loaded with chondrocyte suspension containing chondrocytes, cartilage gel or cartilage sheet pieces.

7. The tissue engineering scaffold of claim 6, wherein the concentration (density) of chondrocytes in the chondrocyte suspension is 1.0×10⁸ cells/ml-10×10⁸ cells/ml.

8. A method for preparing the tissue engineering scaffold of claim 1, which comprises the steps:
(i) preparing a bio-gel solution and place it in a centrifuge tube;
(ii) placing the hard large aperture frame structure in the centrifuge tube containing the bio-gel solution and centrifuging;
(iii) refrigerating the centrifuge tube after centrifugation, taking out the contents for freezing to obtain a bio-gel frame structure complex;
(iv) freeze-drying the bio-gel frame structure complex in vacuum to obtain a freeze-dried bio-gel frame structure complex;
(v) using chemical crosslinking agents to crosslink the freeze-dried bio-gel frame structure complex to obtain a crosslinked bio-gel frame structure complex;
(vi) rinsing the crosslinked bio-gel frame structure complex with deionized water and freeze-drying in vacuum to obtain the tissue engineering scaffold.

9. Use of the tissue engineering scaffold of claim 1 for preparing a medical product for repairing hard tissue defects.

10. The use of claim 9, wherein the hard tissue defects include joint defect, maxillofacial cartilage and related hard tissue defect, nasal septum defect, and a combination thereof.
